(19) United States
(12) Patent Application Publication
(10) Pub. No.: US 2002/0085948 A1
(43) Pub. Date: Jul. 4, 2002

(54) Method and system for circulating ozone using electrophoresis in a sanitizing device

(76) Inventor: Fred J. Conforti, Wheaton, IL (US)

Correspondence Address: Joseph M. Barich, McAndrews, Held & Malloy, Ltd, 34th Floor, 500 West Madison Street, Chicago, IL, 60661, US

(21) Appl. No.: 09/753,106

(22) Filed: Jan. 2, 2001

(51) Int. Cl. A61L 2/20; A61L 9/015; B01J 19/08; C01B 13/11

(52) U.S. Cl. 422/28; 422/29; 422/186.12; 204/176

(57) ABSTRACT

A sanitizing device employs electrophoresis to circulate ozone formed by an ozone generator to sanitize a household item within an interior-sanitizing chamber of the sanitizing device. The electrophoretic movement of air increases the efficiency of ozone generation by the ozone generator. The electrophoretic generator includes two electrodes and a half wave rectifying diode. The electrophoretic generator is connected to a transformer that supplies high voltage pulses to the electrophoretic and ozone generators. The voltage pulse at one of the electrophoretic generator electrodes is half wave rectified by the half wave rectifying diode. The resulting voltage difference between the two electrodes causes a pulsating electric field between the two electrodes. Electrophoretic movement of air is thereby induced, and ozone produced by the ozone generator is circulated within and substantially throughout the sanitizing device. The ozone thus circulated sanitizes an item placed within the sanitizing chamber of the device.

### FIELD OF THE INVENTION

The present sanitizing device employing electrophoretic airflow generation relates to a method and system for generating airflow in a sanitizing device using electrophoresis. More particularly, the present invention relates to a method and system for using electrophoresis to generate airflow to circulate ozone in a sealed sanitizing device to sanitize microbe-laden objects and to increase the efficiency of the ozone generator.

### BACKGROUND OF THE INVENTION

Microbes such as germs, bacteria and viruses may cause millions of illnesses or deaths each year. Various household items may harbor these microbes without the owner even knowing of their harmful effects or existence. A recent study conducted by the University of Arizona demonstrated that microbe levels are particularly high in kitchens and bathrooms. The University of Arizona study included an examination of 500 kitchen sponges and found that one quarter of the sponges tested positive for salmonella or staphylococci-two leading causes of food-borne illness in the U.S. Removing microbes such as salmonella and staphylococci from household items represents an important step in reducing the illnesses that may be caused by these microbes.

Current methods of removing microbes from items such as kitchen sponges and toothbrushes include: washing the household items in dishwashers or clothes washers, microwaving or cleaning the items in boiling water or soaking them in alcohol. These methods, however, may be time consuming and some household items may not be durable enough to withstand frequent cleaning. Also, cleaning may be expensive when reagents such as alcohol are employed in the cleaning process.

An alternative method of sterilizing household items is through the use of ozone, or “Activated Oxygen.” Ozone is an unstable gas that typically degrades to oxygen gas in the presence of air, heat, or water. Ozone typically also destroys microbes. In 1991, the Environmental Protection Agency designated ozone as the most effective primary disinfectant available for drinking water. Ozone is currently used in over 200 cities and over 3,000 state-of-the-art municipal water treatment systems around the world for treating water. Ozone is also used to sterilize medical and dental instruments. Ozone may typically be produced commercially through the use of ultraviolet light or by a corona discharge apparatus.

The corona discharge apparatus derives its name from the glow of a high voltage electric field interacting with oxygen containing gas such as air or pure oxygen. The corona discharge typically generates ozone. A corona discharge apparatus typically includes two electrodes connected to opposing ends of an electrical transformer and a dielectric material placed between the electrodes. Voltage pulses are induced between the two electrodes. Oxygen-containing gas, such as air or pure oxygen, may be passed between the electrodes. Due to interactions between the oxygen containing gas and the high voltage field, a corona discharge is typically formed. The voltage pulses interact with the natural oxygen molecule splitting the natural oxygen into separate, highly active atoms. Some of the highly active atoms may combine with the nearest oxygen molecule to form ozone.

Ozone is typically unstable and seeks equilibrium by reacting with surrounding oxygen molecules or microbes. Because ozone may seek equilibrium by reacting with microbes, ozone may be used to sanitize items such as medical or dental instruments. As ozone is produced, the ozone molecules typically circulate and come into contact with the object being sanitized. Seeking equilibrium, the extra oxygen atom typically breaks through the microbe, leaving behind oxygen gas. Microbes are typically destroyed by a process of cell lysing. That is, microbes are typically destroyed by extra oxygen atoms breaking through the cell walls of the microbes. The extra oxygen atom contained in the ozone typically penetrates the cell wall thereby dispersing the cytoplasm of the cell, prohibiting further reactivation, and thus killing the microbe. The rate of sterilization within the sterilization device typically depends on the concentration of ozone surrounding the item being sanitized.

Some devices use diffusion to circulate ozone. Typically, ozone diffuses slowly; thus, reliance on ozone diffusion alone to sterilize an item may yield prohibitively long sterilization times.

Various ways to increase circulation within a sanitizing device exist. For example, a fan may be employed to increase the rate of circulation of air within the sanitizing device.

Electrophoresis is the migration of charged colloidal particles or charged molecules through a fluid, such as air, under the influence of an applied electric field. Typically, to induce electrophoresis in the fluid, electrodes are immersed in the fluid and an electric field is induced across the electrodes. The charged molecules of the fluid may be attracted or repulsed by the electric field to induce motion in the molecules.

U.S. Pat. No. 4,789,801 issued to Jimmy L. Lee on Dec. 6, 1988 (the '801 patent) illustrates several examples of electrokinetic transducers. FIG. 2 of the '801 patent shows an electrokinetic fan and negative ion generator. The electrokinetic fan and negative ion generator of the '801 patent include an electrode array pair configuration. One of these pairs includes at least four electrodes while the other pair includes at least three electrodes. The '801 patent, however, illustrates electrokinetic transducers and devices that produce minimal amounts of ozone. An electrokinetic fan used for circulating air sometimes generates ozone. One of the important aspects of the electrokinetic fan and negative ion generator of the '801 patent, and the '801 patent in general, is that the '801 patent focuses on minimizing ozone production. Additionally, sterilization is not an objective of the '801 patent.

Alternatively, several different methods may be employed to increase the rate of air circulation. These methods include the use of fans, piezoelectric pumps, and even gravity. These methods, however, may prove too costly, slow or impractical.

To generate commercially usable levels of ozone, present systems often require large, complicated machinery to convert pure oxygen into ozone. The use of these systems within the home is undesirable for many reasons. First, the size of present systems often precludes their use within the home. Second, the amount of ozone produced by these systems typically exceeds the demand for domestic use. Third, the dangers associated with storing a flammable gas such as oxygen in the home may prohibit the use of such systems within the home.

Thus, a need has long existed for a method and system of safely and efficiently removing microbes from household items such as kitchen sponges and toothbrushes. A need has also existed for:

(1) an inexpensive method and system of sanitizing household items;

(2) a smaller and more practical sanitizing device designed for household items;

(3) a better ozone generation system;

(4) a method and system for safely and efficiently circulating ozone;

(5) a more cost effective sanitizing device;

(6) a sanitizing device that is less expensive to manufacture and operate than commercial sanitizing devices; and

(7) a rapid and efficient sanitizing device.

### SUMMARY OF THE INVENTION

The present method and system circulates an airborne sanitizing agent using electrophoresis in a sanitizing device. The airborne sanitizing agent is propelled by the electrophoretic movement of air thereby making the airborne sanitizing agent generator more efficient. Typically, the airborne sanitizing agent is ozone. The method and system for circulating the airborne sanitizing agent includes an ozone generator for generating ozone and an electrophoretic generator circulating the ozone within the sanitizing chamber of the sanitizing device.

The electrophoretic generator increases the efficiency of the ozone generator by electophoretically moving air over the ozone generator, thus moving ozone away from the ozone generator and presenting fresh air. The fresh air is then ozonated in the same way, thus generating more ozone. The electrophoretic generator includes at least two electrodes. Voltage is supplied to the electrodes. The voltage supplied to one of the electrodes is half wave rectified by a half wave rectifying diode. Alternatively, the diode is not necessary if the secondary of the transformer supplies a voltage pulse higher in one polarity. That is, the transformer may supply a +20 kV and a −10 kv pulse. The difference in voltage between the two electrodes causes electrophoretic movement of air. The electrophoretic movement of air, directed by a tube-like element, typically increases the amount of ozone generated and the circulation of ozone generated by the ozone generator.

These and other features of the present method and system are discussed in the following detailed Description of the preferred embodiments of the present sanitizing device employing electrophoretic airflow generation.

### BRIEF DESCRIPTION OF THE DRAWINGS

FIG. 1 is an exploded perspective view of a preferred sanitizing device 100 employing electrophoretic airflow generation;

FIG. 2 illustrates a circuit diagram 200 of the sanitizing device 100 illustrated in FIG. 1; and

FIG. 3 illustrates several high voltage pulses 300 of a preferred embodiment of the present sanitizing device employing electrophoretic airflow generation.

### DETAILED DESCRIPTION OF PREFERRED EMBODIMENT(S)

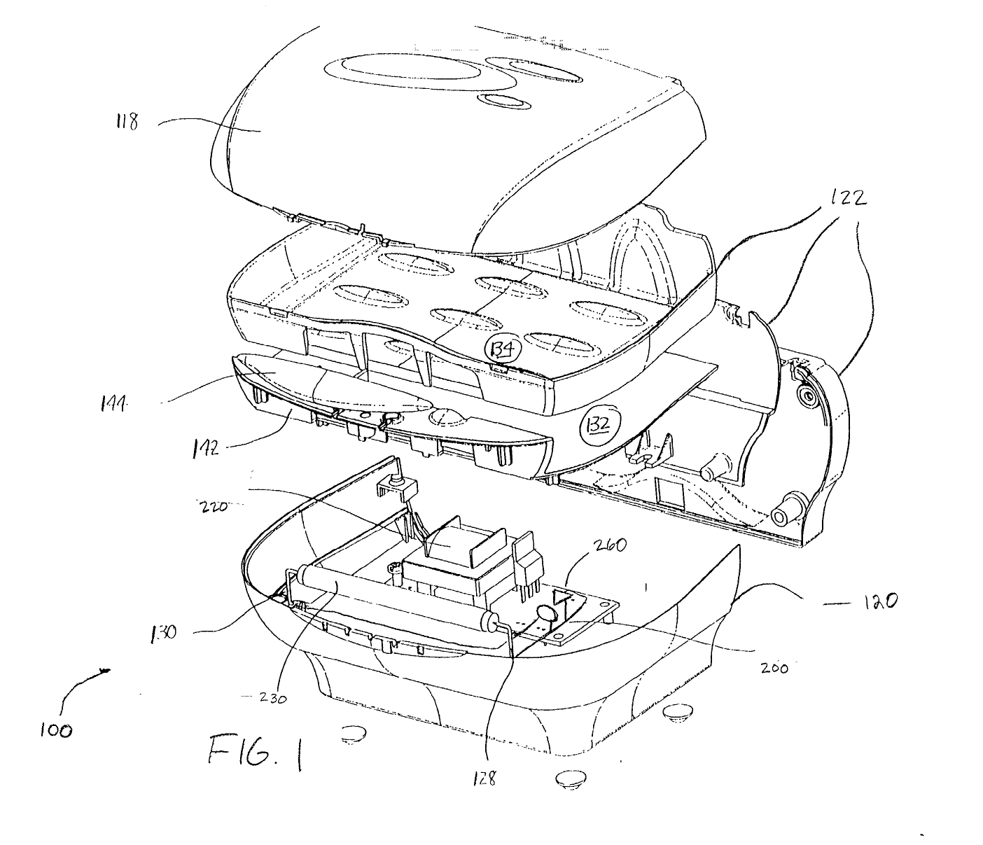

FIG. 1 shows an exploded perspective view of a preferred embodiment of the present sanitizing device 100, which employs electrophoretic airflow generation. The sanitizing device 100 includes a lid 118, a sanitizing device base 120, and upper portions 122.

The sanitizing device base 120 includes a circuit board 200. The circuit board 200 includes a transformer 220, an ozone generator 230, an electrophoretic generator 260, and electrical leads 128 and 130. The upper portions 122 include a divider 132, a sanitizing chamber 134, a slotted wall 142, and a latch 144. Various aspects of the sanitizing device 100 may be further disclosed in U.S. Provisional Patent Application Serial No. 60/141,646 filed Jun. 29, 1999, entitled “Ozone Generator”, which is incorporated by reference herein in its entirety; U.S. Provisional Patent Application Serial No. 60/179,211 filed Jan. 31, 2000, entitled “Ozone Generator”, which is incorporated by reference herein in its entirety; and U.S. Pat. No. 09/607,016 filed Jun. 29, 2000 entitled “Method and Apparatus for Using Ozone to Sanitize Household Objects”, which is also incorporated by reference herein in its entirety.

The divider 132 is connected to the sanitizing device base 120 and to the upper portions 122. The sanitizing chamber 134 of the upper portions 122 rests on top of the divider 132. The slotted wall 142 is formed in the divider 132 and the sanitizing chamber 134. The latch 144 is on the divider 132. The lid 118 is connected to the upper portions 122 and, when closed, the latch 144 latches the lid 118 to the divider 132. The sanitizing device base 120 is also connected to the upper portions 122. In a preferred embodiment of the present sanitizing device employing electrophoretic airflow generation, the base 120 of the sanitizing device is separated from the sanitizing chamber 134 by the divider 132. The divider 132 protects against the ozone produced by the ozone generator 230 and circulated by the electrophoretic generator 260 from entering the sanitizing device base 120 and corroding the electrical components of the circuit board 200.

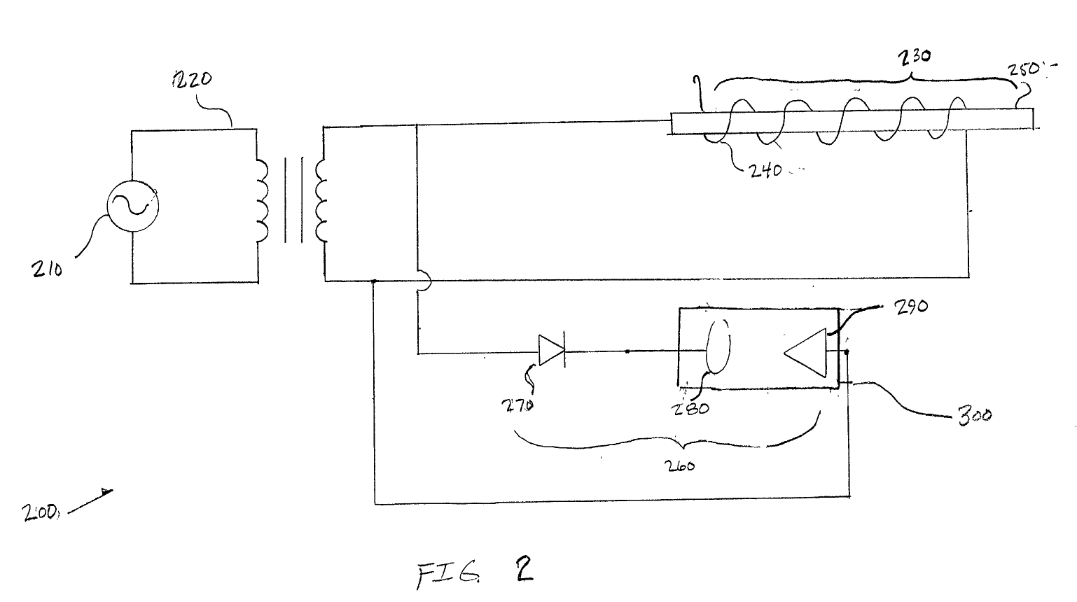

The transformer 220 of the circuit board 200 includes a connection to an external power supply (not shown). The transformer 220 is further connected to the electrophoretic generator 260 and the ozone generator 230. The operation of the circuit board 200 is further described with respect to FIG. 2 below.

The upper portions 122 house a sanitizing chamber 134 that may receive a variety of household goods including a sponge or a toothbrush. The sanitizing tray 134 may be contoured to fit a variety of household goods, such as a sponge or a toothbrush. In operation, a household item to be sanitized is placed in the sanitizing device 100.

In order to place a household item into the sanitizing device 100, the user opens the lid 118 to gain access to the sanitizing chamber 134. After the household item is placed in the sanitizing chamber 134 of the sanitizing device 100, the lid 118 should be in the closed position to prevent any leakage of ozone into the surrounding environment outside the sanitizing device 100. Because the lid 118 remains closed during the sanitizing process, the ozone generator 230 utilizes the air within the sanitizing device 100 to produce ozone. Alternatively, the sanitizing device 100 may have vents to allow for the input of an oxygen-containing gas.

The ozone generated by the ozone generator 230 enters the sanitizing chamber 134 through the slotted wall 142. The electrophoretic generator 260 circulates the ozone produced by the ozone generator 230 within and substantially throughout the sanitizing chamber 134. Ozone produced by the ozone generator 230 and circulated by the electrophoretic generator 260 within the sanitizing chamber 134 comes into contact with the household item being sanitized in the sanitizing chamber 134. The ozone begins to destroy the microbes located on the surface, as well as within the pores (if any), of the household item being sanitized in the sanitizing chamber 134. After sufficient sanitizing time, the sanitizing device 100 is disconnected from the power supply (not shown), or alternatively, the power supply is switched to the off position, and the lid 118 is opened. The object within the sanitizing chamber 134 is then removed. The lid 118 is then closed.

As the ozone generator 230 produces ozone, the ozone generation process is typically rapid and exothermic. Because this reaction is typically rapid and exothermic, the ozone generator 230 and the electrophoretic generator 260 may become hot to the touch. One embodiment of the present sanitizing device employing electrophoretic airflow generation forms a slotted wall 142 between the ozone generator 230. This configuration typically provides protection from the heated ozone generator 230 while allowing the ozone formed to circulate through the slotted wall 142 and into the sanitizing chamber 134.

As an alternative to the ozone generator 230 described in FIG. 1, ozone may be produced in several different ways including using either an ultraviolet or corona discharge generator. A preferred embodiment of the present sanitizing device employing electrophoretic airflow generation utilizes a corona discharge. Generation of ozone through the corona discharge process, including its preferred embodiment, is further disclosed as part of U.S. Provisional Patent Application Serial No. 60/179,211 filed Jan. 31, 2000, entitled “Ozone Generator”, which is incorporated by reference herein in its entirety; U.S. Provisional Patent Application No. 60/179,211 filed Jan. 31, 2000, entitled “Ozone Generator”, which is incorporated by reference herein in its entirety; and U.S. Pat. No. 09/607,052 filed Jun. 29, 2000, entitled “Corona Discharge Ozone Generator with Insulator-Coated Conductors”, which is incorporated by reference herein in its entirety.

Alternatively, the sanitizing device 100 may include various shapes, sizes, vents and configurations to accommodate particular household items. Also, the sanitizing device 100 may include additional ozone generators 230 for increased ozone production. The sanitizing device 100 may also include additional electrophoretic generators 260 for increased ozone circulation.

FIG. 2 illustrates a circuit diagram 200 of the sanitizing device 100, referred to in FIG. 1 above, according to a preferred embodiment of the present sanitizing device employing electrophoretic airflow generation. The circuit diagram 200 includes a voltage generator 210, a transformer 220, an ozone generator 230 and an electrophoretic generator 260, and a flow tube 300. The ozone generator 230 includes a coiled conductor 240 and a conducting core 250. The electrophoretic generator 260 includes a half wave rectifying diode 270, a ring electrode 280 and a pointed electrode 290.

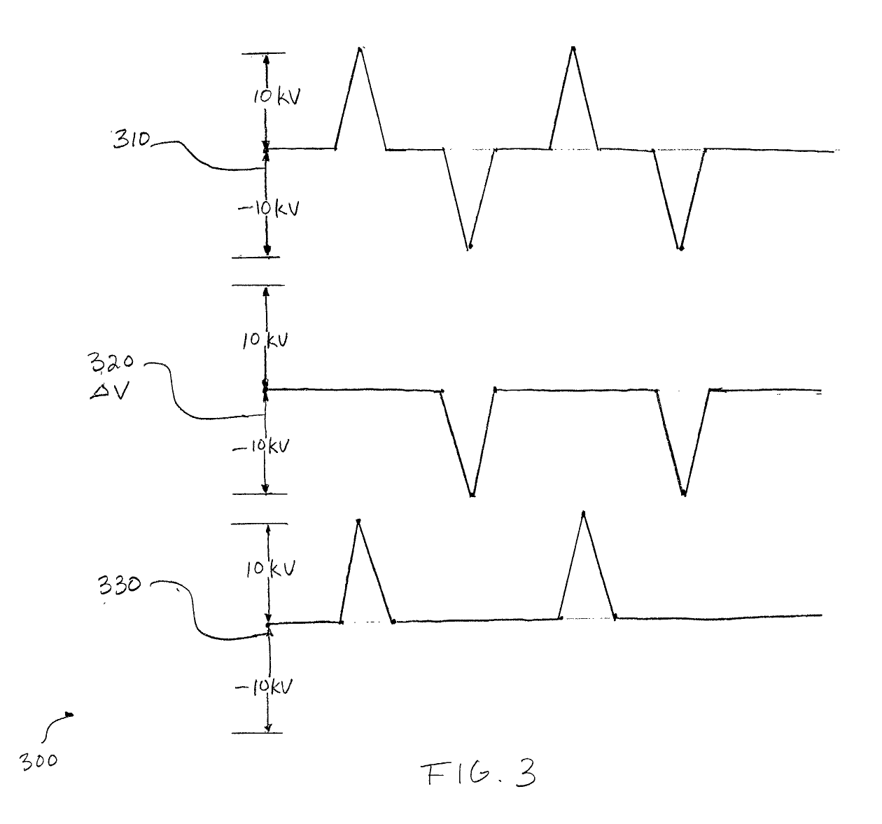

The voltage generator 210 may be an external power source such as an electrical outlet (not shown) supplying, for example, standard wall voltage. The voltage generator 210 is attached to the transformer 220. The transformer 220 transforms the wall voltage received from the voltage generator 210 into high voltage pulses. The high voltage pulses are preferably ±10 kV peak pulses as further described below with reference to FIG. 3. The high voltage pulses are supplied from the transformer 220 to both the ozone generator 230 and the electrophoretic generator 260.

The ozone generator 230 includes a conducting core 250 and a coiled conductor 240 encircling the conducting core 250. The coiled conductor 240 is separated from the conducting core 250 by a layer of air. The air between the conducting core 250 and the coiled conductor 240 acts as a dielectric. The ozone generator 230 is connected to the transformer 220. The ozone generator 230 receives the high voltage pulses from the transformer 220 as further described with reference to FIG. 3 below. The high voltage pulses cause a high voltage electric field to appear between the coiled conductor 240 and the conducting core 250. The high voltage electric field causes a portion of the oxygen in the air between the coiled conductor 240 and the conducting core 250 to form ozone.

Electrophoresis, or electrokinetics, may be employed to produce a flow of fluids such as air. The electrophoretic generator 260 receives the high voltage pulses from the transformer 220 as described above. The high voltage pulses are supplied to the half wave rectifying diode 270. The high voltage pulses that are supplied to the ring electrode 280 pass through the half wave rectifying diode 270. The half wave rectifying diode 270 half wave rectifies the high voltage pulses supplied to the half wave rectifying diode 270 by the transformer 220. The half wave rectifying diode 270 half wave rectifies the negative cycle component of the high voltage pulses as shown in FIG. 3 below. That is, the half wave rectifying diode 270 conducts electricity when the input voltage is positive, but does not conduct when the input voltage is negative. Because the electric field between the pointed electrode 290 and the ring electrode 280 has been half wave rectified, a difference in voltage between the pointed electrode 290 and the ring electrode 280 results. The electric field attracts or repulses charged particles of air within the sanitizing device 100. The difference in voltage between the pointed electrode 290 and the ring electrode 280 causes movement of the charged particles of air within the sanitizing device 100.

Alternatively a voltage difference may be achieved via the transformer 220 supplying a voltage pulse, or spike, higher in one polarity. For example, the transformer 220 may supply a +°kV pulse to one electrode and a −10 kV pulse to the other electrode.

The difference in voltage between the ring electrode 280 and the pointed electrode 290 causes a pulsating electric field to exist between the two electrodes. The pulsating electric field typically facilitates movement of air. The pulsating electric field permits charged particles of air to flow past the ring electrode 280. If a constant electric field existed between the ring electrode 280 and the pointed electrode 290, the movement of the charged particles of air would cease at the ring electrode 280. Because the electric field is pulsating, however, the charged particles of air typically flow within the sanitizing device 100.

The electrophoretic movement of air over the ozone generator 230 typically increases the circulation of the ozone generated by the ozone generator 230. Increased circulation of ozone decreases the amount of time to sanitize the item placed within the sanitizing chamber 134 of the sanitizing device 100. The increased circulation of the ozone generated by the ozone generator 230 typically sanitizes items placed within the sanitizing device 100 quickly and efficiently.

Additionally, air circulation increases the efficiency of the ozone generator 230. As the electrophoretic generator 260 moves air over the ozone generator 230, the electrophoretic generator 260 also generates ozone. The movement of air increases the circulation of ozone produced by the ozone generator 230 and the electrophoretic generator 260. Therefore, the efficiency of ozone production is increased by the electrophoresis generator 260 circulating air over the ozone generator 230 and the ozone produced by the electrophoresis generator 260.

The electrophoretic generator 260 is enclosed in an open-ended cylinder of suitable material, such as plastic. Preferably, the electrophoretic generator 230 would be partially enclosed to promote airflow while simultaneously protecting the electrodes from arcing or other hazards associated with high voltages. Also preferably, the ozone generator 230 is not allowed to come into contact with water in order to prevent arcing or other high voltage hazards. The electrophoretic electrodes preferably are mounted in a fixed position at set distances away from each other. The electrophoresis electrodes function as an operative pair.

Alternatively, the electrophoretic electrodes may be different geometric shapes. Different geometric shapes may be employed so that air flows within the sanitizing chamber. Such shapes may include cones, square hoops, or various other geometric shapes.

FIG. 3 illustrates several high voltage pulses 300 of a preferred embodiment of the present sanitizing device employing electrophoretic airflow generation. The high voltage pulses include transformer voltage pulses 310, ring electrode voltage pulses 330, and differential voltage pulses 320 representing the difference between the transformer voltage pulses 310 and the ring electrode voltage pulses 330. The voltage pulses at the ring electrode 330 are half wave rectified as described above by the half wave rectifying diode 270. The transformer voltage pulses 310 are received by the half wave rectifying diode 270 and the pointed electrode 290. The half wave rectifying diode 270 half wave rectifies the transformer voltage pulses 310 so that the ring electrode 280 receives the ring electrode voltage pulses 330. Because the pointed electrode 290 receives the transformer voltage pulses 310, differential pulses 320 result between the transformer voltage pulses 310 of the pointed electrode 290 and the ring electrode voltage pulses 330 of the ring electrode 280.

Alternatively, the transformer voltage pulse 310 may generate a +20 kV pulse and a −10 kV pulse. The difference in voltage magnitude supplied to the electrodes 280, 290 results in a voltage difference between the two electrodes without the use of the half wave rectifying diode 270.

Several alternative embodiments of the sanitizing device 100 may be developed by those skilled in the art. For example, the sanitizing device 100 may include more than one electrophoretic generator 260 for increased circulation within the sanitizing device 100. Additional electrophoretic generators 260 may be mounted in a variety of ways. For example, the electrophoretic generators 260 may be oriented adjacent to each other, or on opposite sides of the ozone generator 230 inside the sanitizing device 100. The number and orientation of the electrophoretic generators 260 may depend on the size of the sanitizing device 100 and the presence and size of the components within the sanitizing device 100.

Also, the electrophoretic generators 260 produce small amounts of ozone. Thus, the ozone produced by the electrophoretic generator 260 adds to the ozone produced by the ozone generator 230 to increase the total amount of ozone in the sanitizing device 100. Alternatively, the structure of the electrophoresis electrodes may change from their preferred pointed and ring embodiment. Also, alternatively, the positioning of the electrophoresis electrodes may depend on the size of the sanitizing device 100, the presence of additional components within the sanitizing device 100, and the desired circulation rate of ozone within the sanitizing device 100. Additionally, the electrophoretic generators 260 of alternative embodiments may include a number of electrodes of various shapes and configurations. Also, the voltage pulses applied to the half wave rectifying diode 270 may increase or decrease from the preferred ±10 kV depending on the transformer 220 or the voltage supply.

While particular elements, embodiments and applications of the present invention have been shown and described, it will be understood, of course, that the invention is not limited thereto since modifications may be made by those skilled in the art, particularly in light of the foregoing teachings. It is therefore contemplated by the appended claims to cover such modifications that incorporate those features coming within the scope of the invention.

What is claimed is:

1. A sanitizing system having a sanitizing chamber, said sanitizing system employing electrophoresis to circulate an airborne sanitizing agent within said sanitizing chamber.

2. The sanitizing device of claim 1 wherein said sanitizing agent is ozone.

3. The sanitizing system of claim 2 wherein said ozone is produced by an ozone generator.

4. The sanitizing system of claim 3 wherein said ozone generator is a corona discharge generator.

5. The sanitizing system of claim 1 wherein said electrophoresis is generated by an electrophoretic generator.

6. The sanitizing system of claim 5 wherein said sanitizing agent is ozone.

7. The sanitizing system of claim 5 further including:
an ozone generator for generating ozone; and
a high voltage transformer supplying high voltage pulses to said ozone generator and said electrophoretic generator.

8. The sanitizing system of claim 5 wherein said electrophoretic generator comprises two electrodes as an operative pair.

9. The sanitizing system of claim 5 wherein said electrophoretic generator comprises multiple electrodes as operative pairs.

10. The electrophoretic generator of claim 8 wherein said electrodes comprise at least one ring electrode and one pointed electrode.

11. The electrophoretic generator of claim 8 wherein said electrodes are of geometric shapes such that air flows within said sanitizing chamber.

12. The electrophoretic generator of claim 8 wherein high voltage pulses are applied across said electrodes.

13. The electrophoresis generator of claim 10 further including a flow tube for directing air flow

14. The electrophoretic generator of claim 11 further comprising a half wave rectifying diode.

15. A sanitizing system having an ozone generation system, said ozone generation system comprising:
(a) an ozone generator for generating ozone; and
(b) an electrophoretic generator for moving air over said ozone generator to increase the efficiency of said ozone generator.

16. The sanitizing system of claim 15 wherein said ozone generator is a corona discharge generator.

17. A sanitizing system having a sanitizing chamber, said sanitizing system comprising:
(a) an ozone generator for generating ozone; and
(b) an electrophoretic generator for circulating said ozone within said sanitizing chamber.

18. The sanitizing system of claim 17 wherein said ozone generator is a corona discharge generator.

19. The sanitizing system of claim 17 wherein said electrophoretic generator comprises two electrodes oriented to produce air movement within said sanitizing chamber.

20. The electrophoretic generator of claim 19 wherein said electrodes comprise at least one ring electrode and one pointed electrode.

21. The electrophoretic generator of claim 19 wherein said electrodes are of geometric shapes such that air flows within said sanitizing chamber.

22. The electrophoretic generator of claim 19 wherein high voltage pulses are applied across said electrodes.

23. The electrophoretic generator of claim 19 further comprising a half wave rectifying diode.

24. The sanitizing system of claim 17 further including a high voltage transformer supplying high voltage pulses to said ozone generator and said electrophoretic generator.

25. A method for circulating an airborne sanitizing agent within a chamber of a sanitizing device comprising the step of using electrophoresis to circulate said airborne sanitizing agent.

26. The method of claim 25 wherein said step of using electrophoresis comprises positioning at least two electrodes within said chamber and inducing a pulsed high voltage electric field between said electrodes.

27. The method of claim 25 wherein said step of utilizing electrophoresis comprises applying high voltage pulses across at least two electrodes.

28. The method of claim 27 further comprising rectifying high voltage pulses applied to one of said electrodes.

29. The method of claim 25 wherein said airborne sanitizing agent is ozone.

30. The method of claim 25 further comprising the step of generating ozone with an ozone generator.

31. The method of claim 30 wherein said step of generating ozone comprises generating ozone with a corona discharge generator.

32. The method of claim 30 further including the step of supplying high voltage pulses to said ozone generator and said electrophoretic generator via a transformer.

33. A method for increasing the efficiency of ozone generation by an ozone generator including the step of moving air over said ozone generator.

34. The method of claim 32 wherein said moving air step includes moving air over said ozone generator with an electrophoresis generator.
